(19) United States
(12) Patent Application Publication
(10) Pub. No.: US 2025/0249267 A1
(43) Pub. Date: Aug. 7, 2025

(54) IMPLANTABLE MEDICAL DEVICE

(71) Applicant: THE ALFRED E. MANN FOUNDATION FOR SCIENTIFIC RESEARCH, VALENCIA, CA (US)

(72) Inventors: Brian Michael Shelton, Ventura, CA (US); Hitesh Shyamkumar Hotlani, Santa Clarita, CA (US); John Rivera, Oxnard, CA (US)

(21) Appl. No.: 18/954,138

(22) Filed: Nov. 20, 2024

Related U.S. Application Data
(60) Provisional application No. 63/550,242, filed on Feb. 6, 2024.

(51) Int. Cl. A61N 1/372

(52) U.S. Cl. CPC A61N 1/37276; A61N 1/37235

(57) ABSTRACT

An implantable medical device (IMD) may include a first transceiver configured to operate in a first advertising mode, whereby the first transceiver transmits a first advertising signal at a first rate; one or more sensors configured to sense first data; a memory storing instructions; and a microcontroller circuit operatively coupled to the first transceiver, the microcontroller circuit being configured, in response to executing the instructions stored in the memory, to selectively cause the first transceiver to begin operating in the first advertising mode based on the first data.

### CROSS-REFERENCE TO RELATED APPLICATION

This application claims priority to and the benefit of U.S. Provisional Patent Application No. 63/550,242, filed on Feb. 6, 2024, the entire content of which is hereby incorporated by reference.

### BACKGROUND

This background sections is provided only for purposes of introducing certain background material relating to the present disclosure and, thus, is not an admission of prior art.

Implantable medical devices (IMDs) can be implanted in a patient and used for various medical purposes, such as sensing physiological data of the patient or providing stimulation to biological tissue to treat one or more medical conditions. Some IMDs only occasionally need to wirelessly communicate with external devices. For example, a stimulator for under-active bladder (UAB) may only need to be used when the patient needs to eliminate urinary waste, and an implantable pulse generator (IPG) may be able to provide stimulation automatically with only occasional patient intervention. Some IMDs with radios connectable to the outside world (e.g., to radios of external devices) would experience significant and unnecessary battery drain if its radio is always on and using energy to search for other devices to connect with or to transmit advertising signals for other devices to discover. It can therefore be desirable to reduce the energy consumption of the IMD's radio at times when communication between the IMD and external devices is not needed or desired. It is in view of this technical background that the present disclosure is provided.

### SUMMARY

This Summary section introduces some features of nonlimiting and non-exhaustive examples of the present disclosure, and is not intended to limit the scope of the claims.

According to an aspect, the technology relates to an implantable medical device (IMD), including: a first transceiver configured to operate in a first advertising mode, whereby the first transceiver transmits a first advertising signal at a first rate; one or more sensors configured to sense first data; a memory storing instructions; and a microcontroller circuit operatively coupled to the first transceiver, the microcontroller circuit being configured, in response to executing the instructions stored in the memory, to selectively cause the first transceiver to begin operating in the first advertising mode based on the first data.

In some examples, the microcontroller circuit is configured to selectively cause the first transceiver to operate: in the first advertising mode; or in a second advertising mode, whereby the first transceiver does not transmit an advertising signal or transmits the first advertising signal at a second rate less than the first rate.

In some examples, the IMD includes: an implantable pulse generator (IPG) including the first transceiver, the microcontroller circuit, the memory, and a driver configured to generate an electrical current; a lead electrically coupled to the IPG; and a stimulation electrode on the lead and electrically coupled to the driver through the lead, wherein the microcontroller circuit is operatively coupled to the driver and is configured to cause the driver to generate the electrical current while the first transceiver operates in the second advertising mode.

In some examples, a medical system includes: the IMD; and an external device including a second transceiver configured to operate in a first searching mode, whereby the second transceiver searches for an advertising signal, wherein the IMD and the external device are configured to establish a communication channel between the IMD and the external device in response to the second transceiver receiving the first advertising signal and transmitting a reply signal to the first transceiver.

In some examples, the medical system is configured to switch between operating in: an IMD peripheral mode, whereby the first transceiver operates in the first advertising mode and the second transceiver operates in the first searching mode; or an IMD central mode, whereby the first transceiver operates in a second searching mode and the second transceiver operates in a second advertising mode, wherein the first transceiver is configured to search for an advertising signal when operating in the second searching mode, and wherein the second transceiver is configured to transmit second advertising signals when operating in the second advertising mode.

In some examples, the first data includes physiological data, and wherein the microcontroller circuit is configured, when the first transceiver operates in the second searching mode, to cause the first transceiver to search for the advertising signal in response to the microcontroller circuit determining, based on the physiological data, that a physiological condition has occurred or is present.

In some examples, the first transceiver includes a first Bluetooth radio, and the external device includes a second Bluetooth radio.

In some examples, the first data includes physiological data, and the microcontroller circuit is configured to selectively cause the first transceiver to begin operating in the first advertising mode in response to determining, based on the physiological data, that a physiological condition has occurred or is present.

In some examples, the one or more sensors include an electroencephalography (EEG) sensor.

In some examples, the one or more sensors include an electrocardiography (ECG) sensor.

In some examples, the physiological condition includes a seizure.

In some examples, the physiological condition includes an apneic event.

In some examples, the microcontroller circuit is configured to selectively cause the first transceiver to begin operating in the first advertising mode in response to identifying a wake-up signal in the first data.

In some examples, the one or more sensors include at least one of an inertial measurement unit (IMU) or an accelerometer.

In some examples, identifying the wake-up signal includes determining, based on the first data, that a first sequence of taps has occurred against a body of a patient that the IMD is implanted in.

In some examples, the microcontroller circuit is configured to selectively cause the first transceiver to begin operating in the first advertising mode in response to determining, based on the first data, that a patient that the IMD is implanted in is in an upright position or a horizontal position.

In some examples, the one or more sensors include at least one of an inertial measurement unit (IMU) or an accelerometer.

In some examples, the microcontroller circuit is configured to selectively cause the first transceiver to begin operating in the first advertising mode in response to determining that the patient has moved between the horizontal position and the upright position.

In some examples, the microcontroller circuit is configured to cause the first transceiver to begin a communication shut-down operation in response to determining that the patient has moved from the upright position to the horizontal position, the communication shut-down operation including the first transceiver stopping its operating in the first advertisement mode within a set first time period after the microcontroller circuit determines that the patient has moved from the upright position to the horizontal position.

In some examples, the microcontroller circuit is configured to cause the first transceiver to begin a communication activation operation in response to determining that the patient has moved from the horizontal position to the upright position, the communication activation operation including the first transceiver beginning its operating in the first advertising mode within a first set time period after the microcontroller circuit determines that the patient has transitioned from the horizontal position to the upright position.

In some examples, the communication activation operation includes causing the first transceiver to operate in the first advertising mode for a second time period, and to stop operating in the first advertising mode after the second time period. In some examples, the first data includes a respiration level.

In some examples, the microcontroller circuit is configured to selectively cause the first transceiver to begin operating in the first advertising mode based on whether the respiration level is below a threshold respiration level and whether the patient is in the horizontal position.

In some examples, the IMD includes a clock configured to determine a time of day, wherein the microcontroller circuit is configured to collect second data about time(s) of day when the patient is in the horizontal position, and to selectively cause the first transceiver to begin operating in the first advertising mode based on the time of day and the second data.

In some examples, the IMD includes a clock configured to determine a time of day, wherein the microcontroller circuit is configured to selectively cause the first transceiver to operate in the first advertising mode or in a second advertising mode, whereby the first transceiver does not transmit an advertising signal or transmits the first advertising signal at a second rate less than the first rate, and wherein the microcontroller circuit is configured to collect second data about time(s) of day when the first transceiver is operated in the first advertising mode, and to selectively cause the first transceiver to begin operating in the first advertising mode based on the time of day and the second data.

According to another aspect, the technology relates to an implantable medical device (IMD), including: a first transceiver configured to operate in a high-power connectable mode and in a low-power mode; a memory storing instructions; and a microcontroller circuit operatively coupled to the first transceiver, the microcontroller circuit being configured, in response to executing the instructions stored in the memory, to selectively cause the first transceiver to operate in the high-power connectable mode or in the low-power mode.

In some examples, the first transceiver is configured, when operating in the high-power connectable mode, to transmit a first advertising signal at a first rate, and wherein the first transceiver is configured, when operating in the low-power mode, to not transmit an advertising signal or to transmit the first advertising signal at a second rate less than the first rate.

In some examples, the first transceiver is configured, when operating in the high-power connectable mode, to search for advertising signals and to use energy at a first rate, and the first transceiver is configured, when operating in the low-power mode, to not search for advertising signals or to search for advertising signals while using energy at a second rate less than the first rate.

In some examples, the microcontroller circuit is configured to cause the IMD to perform one or more core operations while the first transceiver operates in the low-power mode, the one or more core operations including at least one of providing stimulation, sensing physiological data, or discharging a medical substance.

In some examples, the IMD includes: an implantable pulse generator (IPG) including the first transceiver, the microcontroller circuit, the memory, and a driver configured to generate an electrical current; a lead electrically coupled to the IPG; and a stimulation electrode on the lead and electrically coupled to the driver.

In some examples, the IMD includes one or more sensors, wherein the microcontroller circuit is configured to cause the first transceiver to transition from operating in the low-power mode to operating in the high-power connectable mode in response to determining, based on physiological data sensed by the one or more sensors, that a physiological condition has occurred or is present.

In some examples, the one or more sensors include at least one of an electroencephalography (EEG) sensor or an electrocardiography (ECG) sensor.

In some examples, the physiological condition includes at least one of a seizure or an apneic event.

In some examples, the IMD includes one or more sensors, wherein the microcontroller circuit is configured to cause the first transceiver to transition from operating in the low-power mode to operating in the high-power connectable mode in response to identifying a wake-up signal in data sensed by the one or more sensors.

In some examples, the one or more sensors include at least one of an inertial measurement unit or an accelerometer.

In some examples, the identifying the wake-up signal includes determining that a first sequence of taps has occurred against a body of a patient that the IMD is implanted in.

In some examples, the IMD includes: an implantable pulse generator (IPG) including the first transceiver, the microcontroller circuit, the memory, and a driver configured to controllably provide an electrical current; a lead electrically coupled to the IPG; and a stimulation electrode on the lead and electrically coupled to the driver, wherein the microcontroller circuit is operatively coupled to the driver and configured to cause the driver to provide the electrical current to the stimulation electrode in response to determining that the first sequence of taps has occurred against the body.

In some examples, the IMD includes: an implantable pulse generator (IPG) including the first transceiver, the microcontroller circuit, the memory, and a driver configured to controllably provide an electrical current; a lead electrically coupled to the IPG; and a stimulation electrode on the lead and electrically coupled to the driver, wherein the microcontroller circuit is operatively coupled to the driver and configured to cause the driver to provide the electrical current in response to determining that a second sequence of taps against the body, different from the first sequence of taps, has occurred.

In some examples, the IMD includes one or more sensors, wherein the microcontroller circuit is configured to cause the first transceiver to transition between operating in the low-power mode and operating in the high-power connectable mode in response to determining, based on data sensed by the one or more sensors, that a patient that the IMD is implanted in has moved between a horizontal position and an upright position.

In some examples, the one or more sensors include at least one of an inertial measurement unit or an accelerometer.

In some examples, the microcontroller circuit is configured, in response to determining that the patient has moved from the upright position to the horizontal position, to cause the first transceiver to operate in the high-power connectable mode for a first time period and to transition from operating in the high-power connectable mode to operating in the low-power mode after the first time period.

In some examples, the microcontroller circuit is configured, in response to determining that the patient has moved from the horizontal position to the upright position, to cause the first transceiver to transition from operating in the low-power mode to operating in the high-power connectable mode.

In some examples, the IMD includes a clock configured to determine a time of day, wherein the microcontroller circuit is configured to selectively cause the first transceiver to operate in the high-power connectable mode based on the time of day.

In some examples, the microcontroller circuit is configured to collect second data about what time(s) of day the first transceiver is operated in the high-power connectable mode, and to selectively cause the first transceiver to operate in the high-power connectable mode based on the second data.

In some examples, the IMD includes one or more sensors, wherein the microcontroller circuit is configured to selectively cause the first transceiver to operate in the high-power connectable mode in response to at least one of: determining a physiological condition based on physiological data sensed by the one or more sensors; identifying a wake-up signal in data sensed by the one or more sensors; or determining that a set change has occurred in a position of a patient's body that the IMD is implanted in.

In some examples, a medical system includes: the IMD; and an external device including a second transceiver configured to establish a communication channel with the first transceiver when the first transceiver operates in the high-power connectable mode.

In some examples, the first and second transceivers are not configured to establish the communication channel when the first transceiver operates in the low-power mode.

According to another aspect, the technology relates to a method for operating an implantable medical device (IMD) including a first transceiver, the method including: operating the first transceiver in a low-power mode; determining that a communication threshold condition has occurred or is present; and in response to determining that the communication threshold event has occurred or is present, operating the first transceiver in a high-power connectable mode, whereby the first transceiver transmits advertising signals at a first rate or searches for advertising signals, the first transceiver uses energy at a higher rate when operating in the high-power connectable mode than when operating in the low-power mode.

In some examples, when the first transceiver operates in the low-power mode, the first transceiver does not transmit advertising signals and does not search for advertising signals.

In some examples, the determining that the communication threshold event has occurred or is present includes determining that a physiological condition has occurred or is present.

In some examples, the determining that the communication threshold event has occurred or is present includes identifying a wake-up signal in data sensed by the IMD.

In some examples, the determining that the communication threshold event has occurred or is present includes determining that a set change has occurred in a position of a patient's body that the IMD is implanted in.

In some examples, the determining that the communication threshold event has occurred or is present includes determining that the time of day is within a set range.

To the extent that they are consistent, features described in this Summary section may be included in any combination.

### BRIEF DESCRIPTION OF THE DRAWINGS

The drawings, together with the specification, illustrate nonlimiting and non-exhaustive examples of the present disclosure.

FIG. 1 illustrates a medical system, including an implantable medical device (IMD) implanted within a patient and an external device, according to some examples.

FIG. 2 schematically illustrates the medical system of FIG. 1 with three examples of the external device, according to some examples.

FIG. 3 illustrates the IMD of the medical system of FIG. 1, according to some examples.

FIG. 4 schematically illustrates the IMD and the external device of the medical system of FIG. 1, according to some examples.

FIG. 5 illustrates the activity levels of the respective transceivers of the IMD and the external device of FIG. 1 over time, according to some examples where the IMD functions as a peripheral device.

FIG. 6 illustrates the activity levels of the respective transceivers of the IMD and the external device of FIG. 1 over time, according to some examples where the IMD functions as a central device.

FIG. 7 illustrates the activity levels of the respective transceivers of the IMD and external device of FIG. 1 over time, according to some examples where the IMD is configured to selectively function as a peripheral device or as a central device.

FIG. 8 illustrates a method for operating an IMD, according to some examples.

### DETAILED DESCRIPTION

Nonlimiting and non-exhaustive example embodiments of an implantable medical device (IMD) that includes a transceiver, and methods for operating a transceiver of an IMD, will now be described with reference to the attached drawings. According to some examples of the present disclosure, the transceiver of an IMD can reduce the amount of energy it uses to transmit advertising signals, or to search for advertising signals, while also reliably communicatively connecting to an external device at time(s) of the day when communication between the IMD and the external device may be needed or desired.

The subject matter of the present disclosure may be embodied in various different forms, and should not be construed as being limited to only the illustrated and described examples herein. Rather, these examples are provided so that this disclosure will be thorough and complete, and will fully convey certain aspects and features of the present disclosure to those skilled in the art. Accordingly, processes, elements, and techniques that are not necessary to those having ordinary skill in the art for a complete understanding of aspects and features of the present disclosure may not be described. Unless otherwise noted, like reference numerals denote like elements throughout the attached drawings and the written description, and thus, redundant descriptions thereof may not be repeated. In the drawings, the relative sizes of elements and regions may be exaggerated for clarity.

It will be understood that, although the terms “first”, “second”, “third”, etc., may be used herein to describe various elements, processes, or other features, these elements, processes, or features should not be limited by these terms. These terms are only used to distinguish one element, process, or feature from another element, process, or feature. Thus, a first element, process, or feature discussed herein could be termed a second element, process, or feature, without departing from the spirit and scope of the present disclosure.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises,” “comprising,” “includes,” and “including,” specify the presence of stated elements, processes, and/or other features, but do not preclude the presence or addition of one or more other elements, processes, and/or features. As used herein, the term “and/or” includes any and all combinations of one or more of the associated listed items. Expressions such as “at least one of,” when preceding a list of elements, modify the entire list of elements and do not modify the individual elements of the list. Further, the use of “may” when describing embodiments of the present disclosure refers to “one or more embodiments of the present disclosure.”

It will be understood that when an element is referred to as being “on”, “connected to”, “coupled to”, “attached to”, or “adjacent to” another element, it can be directly on, connected to, coupled to, attached to, or adjacent to the other element, or one or more intervening element(s) may be present. In contrast, when an element is referred to as being “directly on,” “directly connected to”, “directly coupled to”, “directly attached to”, or “immediately adjacent to” another element, there are no intervening elements present. Similar terms and phrases should be understood in a similar manner to encompass both direct and indirect affiliations between two or more elements being discussed. In addition, it will also be understood that when an element is referred to as being “between” two elements or layers, it can be the only element between the elements, or one or more intervening elements may also be present.

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The electronic or electric devices and/or any other relevant devices or components according to examples of the present disclosure described herein may be implemented utilizing any suitable hardware, firmware (e.g., an application-specific integrated circuit), software, or a combination of software, firmware, and hardware. For example, the various components of these devices may be formed on one integrated circuit (IC) chip or on separate IC chips. Further, the various components of these devices may be implemented on a flexible printed circuit film, a tape carrier package (TCP), a printed circuit board (PCB), or formed on one substrate. Further, the various components of these devices may be a process or thread, running on one or more processors, in one or more computing devices, executing computer program instructions and interacting with other system components for performing the various functionalities described herein. The computer program instructions may be stored in a memory which may be implemented in a computing device using a standard memory device, such as, for example, a random-access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the spirit and scope of the example embodiments of the present disclosure.

Unless otherwise defined, all terms (including technical and scientific terms) used herein have the same meaning as commonly understood by one of ordinary skill in the art to which the present disclosure belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

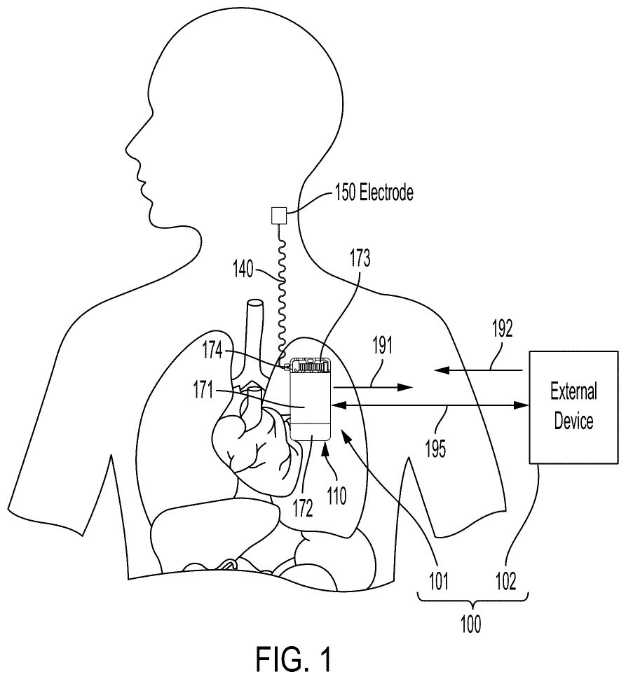

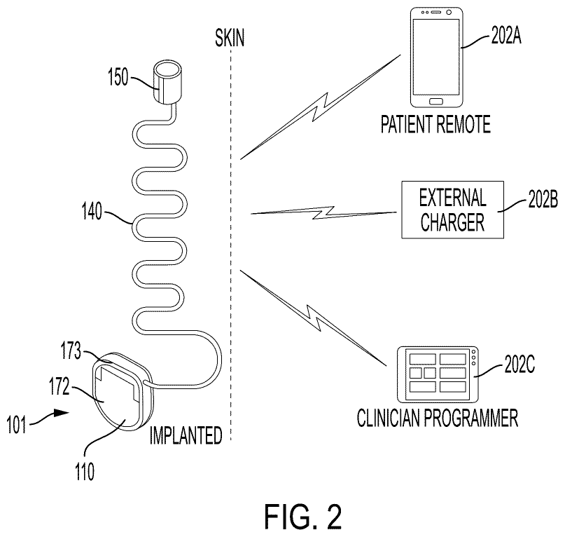

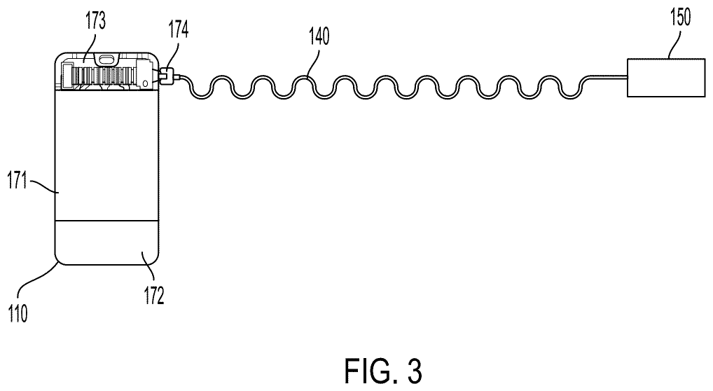

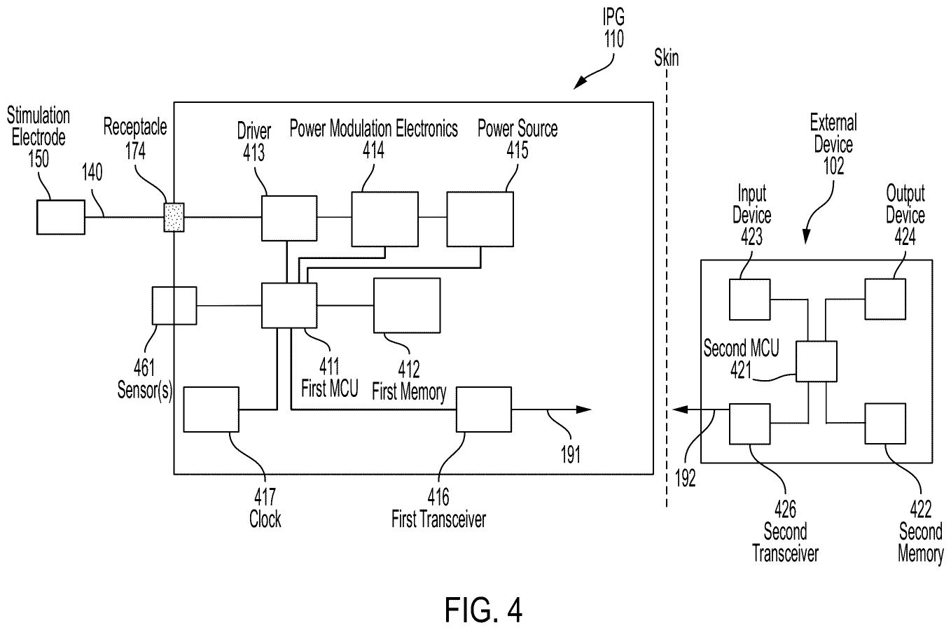

FIG. 1 illustrates a medical system, including an implantable medical device (IMD) 101 implanted within a patient and an external device 102, according to some examples. FIG. 2 schematically illustrates the medical system of FIG. 1 with three examples of the external device 102, according to some examples. FIG. 3 illustrates the IMD 101 of the medical system of FIG. 1, according to some examples. FIG. 4 schematically illustrates the IMD 101 and the external device 102 of the medical system of FIG. 1, according to some examples.

The medical system may include the IMD 101 and the external device 102, which may be configured to wirelessly communicate with each over (e.g., over a radio frequency (RF) channel), and may be configured to controllably establish and disconnect a communication channel 195 between the IMD 101 and the external device 102. In some examples, the IMD 101 and the external device 102 may each include a radio, such as a Bluetooth (e.g., Bluetooth Low Energy) radio, and may be configured to pair with each other to establish the communication channel 195 (e.g., a bi-directional communication channel). Although the technology of the present application will be largely discussed in the context of Bluetooth radios, the present application is not limited thereto, and the technology is applicable to other wirelessly communicable devices that are configured to wirelessly communicate (e.g., configured to advertise, search, and/or establish wireless communication channels) using similar or analogous technology and/or methods as those within the scope of the present application.

The IMD 101 may be configured to selectively configure itself to be in a connectable mode or in a non-connectable mode. In the connectable mode, the IMD 101 may be configured to allow the communication channel 195 to be established, for example, by transmitting advertising signals or searching for transmitting signals from external devices. In the non-connectable mode, the IMD 101 may be unable to allow the communication channel 195 to be established, for example, by not transmitting advertising signals and not searching for transmitting signals from external devices. As explained in more detail below, the IMD 101 uses energy to transmit the advertising signals and to search for advertising signals from other devices. By selectively configuring itself to be in the connectable mode only at set times, as opposed to all the time, the IMD 101 can conserve energy, which can prolong the battery life of the IMD 101 and/or allow a smaller battery to be included in the IMD 101. In some examples, the IMD 101 can also conserve energy by selectively configuring itself to be in a high-power connectable mode or in a low-power connectable mode, where the low-power connectable mode consumes less energy than the high-power connectable mode.

Connecting (e.g., pairing) two Bluetooth devices may generally begin with a first Bluetooth device (e.g., a “peripheral” or “slave” device) transmitting advertising signals (e.g., packets of advertising data) periodically at a set rate. The advertising signals may include, for example, address information of the first Bluetooth device with or without additional information (e.g., the name of the first Bluetooth device). A second Bluetooth device (e.g., a “central” or “master” device) may be in a searching (or discovery) mode, whereby it searches for (e.g., accepts and/or attempts to identify) advertising signals transmitted from other Bluetooth devices. When the second Bluetooth device receives one of the advertising signals from the first Bluetooth device, the second Bluetooth device can transmit a reply signal that includes its own address information with or without additional information (e.g., the name of the second Bluetooth device). This reply signal may also include a request to connect to the first Bluetooth device. Once the first and second Bluetooth devices have each other's address information, they can connect (or pair) with each other through a pairing process to securely establish a communication channel between the first and second Bluetooth devices. The pairing process may include the first and second Bluetooth devices exchanging security keys (e.g., encryption keys) and/or the first Bluetooth device authenticating the second Bluetooth device to confirm that the second Bluetooth device is a device that is authorized to communicate with the first Bluetooth device. In some examples, two devices that have connected via a pairing process that includes the authentication process can store information about each other such that, if the two devices disconnect and subsequently attempt to re-connect, the re-connection can be performed without repeating (e.g., can skip) the authentication process. The communication channel that is established can allow bi-directional communication between the first and second Bluetooth devices. In some examples, the IMD 101 and the external device 102 can connect to each other in this manner. A Bluetooth device expends energy to transmit the advertising signals and to search for advertising signals. By reducing the amount of time that a Bluetooth radio spends transmitting the advertising signals or searching for advertising signals, the Bluetooth device can conserve energy.

The IMD 101 may include any type of IMD device, such as an implantable pulse generator (IPG), a dedicated sensor configured to sense (e.g., detect or measure) physiological data, an IMD configured to controllably discharge a medical substance (e.g., an insulin pump), a defibrillator, etc. The IMD 101 will be primarily described herein as including an implantable pulse generator (IPG) 110 for providing electrical stimulation to treat one or more medical conditions, but this is only an example. The IMD 101 may be configured to perform any combination of one or more core functions, such as providing stimulation, sensing physiological data, and/or discharging a medical substance, etc.

The external device 102 may include any type of device that is configured to wirelessly communicate with the IMD 101 while it is implanted in a patient. For example, the external device 102 may be a device configured to communicate with other devices via an RF radio (e.g., a Bluetooth radio). With reference to FIG. 2, examples of the external device 102 may include a patient remote 202A, an external charger 202B, or a clinician programmer 202C. The patient remote 202A may be a device, such as a smart phone, tablet, computer, or smart watch, that the patient can use to, for example, monitor data collected by the IMD 101 and/or to transmit data (e.g., instructions) to the IMD 101 to control one or more operations of the IMD 101. The external charger 202B may be a device configured to recharge (e.g., inductively recharge) a battery of the IMD 101. The clinician programmer 202C may be a device for a clinician to use to program or control the IMD 101 and/or to receive and review data collected by the IMD 101.

The IMD 101 may include the IPG 110, a lead 140 electrically coupled to the IPG 110, and an electrode 150 (e.g., a stimulation electrode) on the lead 140. The IPG 110 may include a housing 171 (e.g., a metal CAN) configured to house (e.g., contain) at least some of the components of the IPG 110, a ceramic base 172 on one side of the housing 171, and a header 173 at another side (e.g., opposite side) of the housing 171. The header 173 may include, for example, a glass or plastic material and may include a receptacle 174 configured to receive a proximal end of the lead 140. The header 173 may include electrical contact pins configured to respectively provide an electrical connection between an electrical contact on the proximal end of the lead 140 and one or more components of the IPG 110. The ceramic base 172 may be included so that the base end of the IPG 110 is not covered by a conductive material (e.g., a metal). This can allow magnetic fields (e.g., inductive charging fields or communication signals) to more easily enter the housing 171 to interact with components contained within the housing 171 (e.g., a transceiver and/or a charging circuitry) or exit the IPG 110. The IPG 110 may include (e.g., on its external surface) a biocompatible material and may be hermetically sealed to reduce or prevent electrical and/or chemical interactions between tissue around the IPG 110 and components contained within the IPG 110.

Turning to FIG. 4, the IPG 110 may include a power source 415 (e.g., a rechargeable or non-rechargeable battery), a driver 413 configured to generate an electric current, power modulation electronics 414 coupled between the power source 415 and the driver 413, one or more sensors 461, a first transceiver 416, a clock 417, a first memory 412, and a first microcontroller unit (MCU) 411 (e.g., a microcontroller circuit). The first MCU 411 may be operatively connected to at least one of the driver 413, the power modulation electronics 414, the power source 415, the sensor(s) 461, the first transceiver 416, the clock 417, or the first memory 412. The first MCU 411 may be configured to control at least some operations of the of the IMD 101, including any combination of one or more of the operations of the IMD 101 described herein. The first memory 412 may store instructions (e.g., computer readable instructions) that, when accessed and executed by the first MCU 411, cause the first MCU 411 to perform (e.g., via one or more of the components of the IMD 101) any combination of one or more of the operations of the IMD 101 described herein.

The driver 413 may be configured to generate an electrical current (e.g., a stimulation current) and provide the electrical current to the stimulation electrode 150 through the lead 140. The driver 413 may be configured to generate (e.g., under the control of the first MCU 411) the electrical current based on a voltage provided by the power modulation electronics 414. The generated electrical current may include one or more pulses, for example, a pulse train having a set frequency, a set pulse width, a set pulse amplitude, and/or a set duty cycle. The power modulation electronics 414 may be configured to modulate (e.g., under the control of the first MCU 411) a voltage provided by the power source 415 and to provide the modulated voltage to the driver 413. The stimulation electrode 150 may be configured to stimulate specific tissue, such as the vagus nerve, the hypoglossal nerve, other nerve tissue or bundles, the bladder, heart tissue, other organs, etc. The stimulation electrode 150 may include one or more electrode contacts (e.g., individually and differentially addressable sub-electrodes), and the configuration of the stimulation electrode 150 for stimulating specific tissue may include the number, shape, and arrangement of electrode contacts. For example, a cuff electrode configured to stimulate the vagus nerve may include a plurality of electrode contacts on a flexible band that is configured to be wrapped around the vagus nerve.

Providing stimulation via the stimulation electrode 150 may be a core function that the IMD 101 is configured to perform. In some examples, the IMD 101 may additionally or alternatively be configured to perform one or more other core functions, such as sensing one or more physiological parameters or conditions and/or controllably discharging a medical substance. For example, the IMD 101 may include one or more dedicated sensors configured to sense the one or more physiological parameters or conditions and/or may include a pump (e.g., an insulin pump) configured to discharge the medical substance (e.g., insulin). In some examples, the one or more dedicated sensors may be the same as, or separate from, the sensor(s) 461 described in more detail below.

The clock 417 may be configured to determine (e.g., track or monitor) the time of day. This can be advantageous when the first MCU 411 is configured to control certain operations of the IMD 101 based on the time of day.

The first transceiver 416 may be configured to wirelessly transmit a communication signal 191 and wirelessly receive communication signals (e.g., the communication signal 192 described in more detail below). The first transceiver 416 may include a receiver and a transmitter, which may be separate components or may be integrally formed together as a single unit. In some examples, the first transceiver 416 includes an RF radio, for example, a Bluetooth (e.g., a classic Bluetooth or Bluetooth Low Energy) radio.

The external device 102 may include an input device 423, an output device 424, a second transceiver 426, a second memory 422, and a second microcontroller unit (MCU) 421 (e.g., a microcontroller circuit).

The second MCU 421 may be operatively coupled to at least one of the input device 423, the output device 424, the second transceiver 426, or the second memory 422, and may be configured to control at least some operations of the external device 102, including any combination of one or more of the operations of the external device 102 described herein. The second memory 422 may store instructions (e.g., computer readable instructions) that, when accessed and executed by the second MCU 421, cause the second MCU 421 (e.g., via one or more components of the external device 102) to perform one or more operations of the external device 102, including any combination of one or more of the operations described herein.

The second transceiver 426 may be configured to wirelessly transmit the communication signal 192 and to wirelessly receive communication signals (e.g., the communication signal 191). The second transceiver 426 may include a receiver and a transmitter, which may be separate components or may be integrally formed together as a single unit. In some examples, the second transceiver 426 includes an RF radio, for example, a Bluetooth (e.g., a classic Bluetooth or Bluetooth Low Energy) radio. The first and second transceivers 416 and 426 may be configured to connect with each other to establish a bi-directional communication channel 195 so that data can be freely transmitted between the first and second transceivers 416 and 426. As explained above, establishing the bi-directional communication channel 195 may begin with one of the first transceiver 416 or the second transceiver 426 (e.g., the peripheral device) transmitting advertising signals including at least its address information. If the other one of the first transceiver 416 or the second transceiver 426 (e.g., the central device) receives (e.g., discovers) the transmitted advertising signal, it may respond by transmitting a reply signal with at least its own address information and, in some examples, a request to connect. When the first and second transceivers 416 and 426 have each other's address information, they can establish the communication channel 195 via a pairing process, which may include exchanging security keys (e.g., encryption keys) and an authentication process by one or both of the first and second transceivers 416 and 416 to determine if the other device is an authorized device (e.g., a trusted device). As explained in more detail below, the first transceiver 416 of the IMD 101 may be configured to function as either the peripheral device or the central device, and may be configured to selectively function in either role (e.g., to controllably reverse roles).

The input device 423 may include any device configured to receive user input, such as a keyboard, a touch screen, a microphone, etc. User input data received by the input device 423 may be processed by the second MCU 421 and/or transmitted to the IMD 101 via the second transceiver 426. The user input data may include instructions to control one or more operations of the IMD 101 and/or to control one or more operations of the external device 102.

The output device 424 may include any device configured to output information, such as a display screen, a speaker, etc. Information, such as data collected by the IMD 101 and transmitted to the external device 102, may be output via the output device 424.

The first transceiver 416 may be controllably configured to be in a connectable mode, whereby the first transceiver 416 is able to wirelessly connect (e.g., able to establish the communication channel 195) with other devices. The connectable mode may include at least one of an advertising mode or a searching mode. The first transceiver 416 may function as a peripheral device and transmit advertising signals at a set rate when it operates in the advertising mode, and the first transceiver 416 may function as a central device and search for advertising signals when it operates in the searching mode. In some examples, the first transceiver 416 may be configured to operate in only one of the advertising mode or the searching mode. In some other examples, the first transceiver 416 may be selectively operable in either the advertising mode or the searching mode.

When the first transceiver 416 is in the searching mode, it may be configured to receive (e.g., controllably accept) advertising signals and/or identify an advertising signal from among all signals received by the first transceiver 416 (e.g., to filter out noise and/or unwanted signals to identify an advertising signal based on set criteria). For example, the first transceiver 416 may include an antenna that is configured to be selectively turned on and off to controllably accept or refuse incoming signals, and the antenna may be on when the first transceiver 416 is in the searching mode. In some examples, the first transceiver 416 (or the first MCU 411) analyzes signals received by the antenna to identify advertising signals from among the signals received by the antenna when the first transceiver is in the searching mode, and the first transceiver 416 does not analyze the signals when the first transceiver 416 is not in the searching mode. As used herein, an advertising signal may include a packet of information that includes, among other potential information, a manufacturer identification number and/or a device identification number. Each of the manufacturer identification number and the device identification number may, for example, indicate the type of device (e.g., clinician programmer, smart phone, IMD, IPG, etc.) that the transmitting device is and/or the manufacturer of the transmitting device. Filtering out noise and/or unwanted signals may include filtering out signals that are not identified as being associated with a type of device from among one or more specific types of devices (e.g., approved or trusted types of devices) and/or associated with a manufacturer from among one or more specific manufacturers (e.g., approved or trusted manufacturers). This filtering may be performed, for example, by the first MCU 411 and/or by another MCU of the first transceiver 416 (e.g., an MCU integrally formed with, or dedicated to, the first transceiver 416).

In some examples, the first transceiver 416 may be configured to use a single antenna for receiving signals and transmitting advertising signals. In some other examples, the first transceiver 416 includes different antennas respectively for receiving signals and transmitting advertising signals.

In some examples, the first transceiver 416 may be operated in a non-connectable mode, whereby it neither transmits advertising signals nor searches for other devices. The first transceiver 416 may be unable to wirelessly connect (e.g., unable to establish the communication channel 195) with other devices when the first transceiver 416 is in the non-connectable mode. For example, the antenna(s) for receiving signals and transmitting advertising signals may be turned off when the first transceiver 416 is in the non-connectable mode.

As explained in more detail below, the first transceiver 416 may be configured to be controllably switched (e.g., under the control of the first MCU 411) between operating in the connectable and non-connectable modes in response to a communication threshold condition (e.g., event) occurring. Energy is required to operate the first transceiver 416 in the connectable mode, for example, by transmitting the advertising signals or by operating the antenna for receiving signals and analyzing received signals. By configuring the first transceiver 416 to be selectively switched between operating in the connectable and non-connectable modes, the first transceiver 416 can be switched into the connectable mode during times in which it is desirable or expected for the IMD 101 to communicate (e.g., to transmit information to, or receive data or instructions from) with the external device 102. The first transceiver 416 can be switched into the non-connectable mode (or into a lower-power connectable mode) during other times when it is not desirable or expected for the IMD 101 and the external device 102 to communicate with each other.

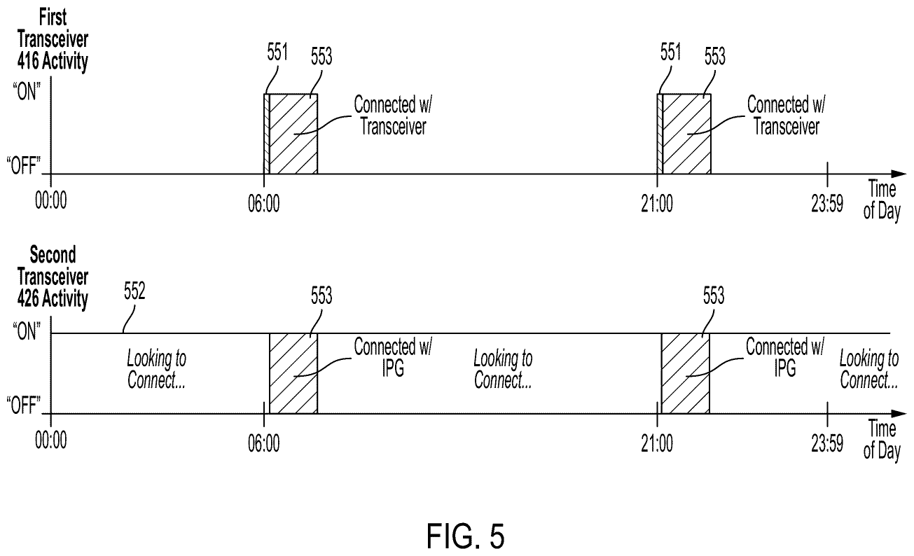

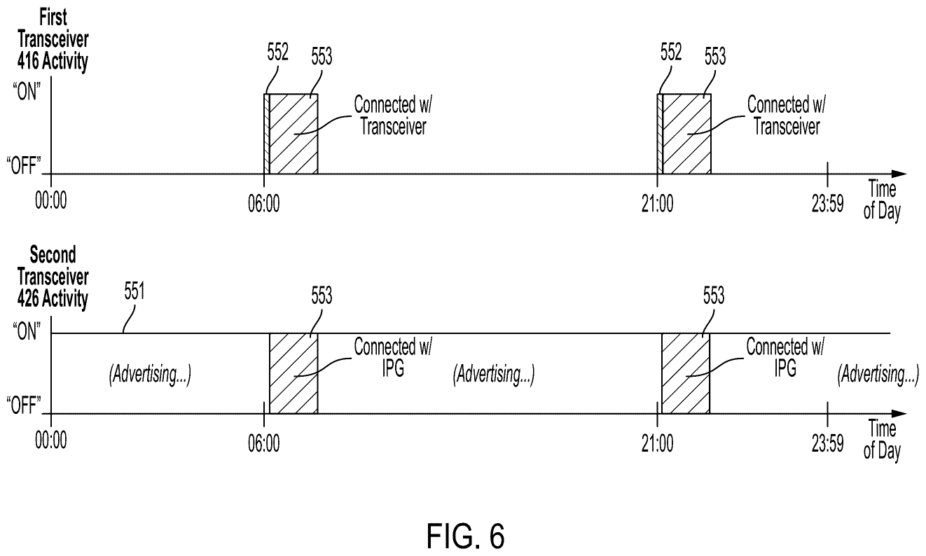

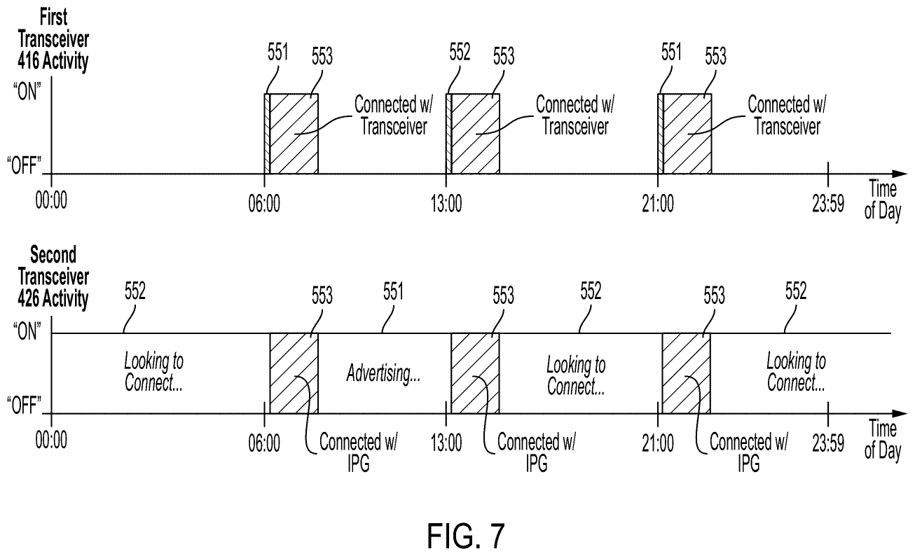

FIG. 5 illustrates the activity levels of the first and second transceivers 416 and 426 respectively of the IMD 101 and the external device 102 over time, according to some examples where the IMD 101 functions as a peripheral device. FIG. 6 illustrates the activity levels of the first and second transceivers 416 and 426 of the IMD 101 and external device 102 over time, according to some examples where the IMD 101 functions as a central device. FIG. 7 illustrates the activity levels of the first and second transceivers 416 and 426 respectively of the IMD 101 and external device 102 over time, according to some examples where the IMD 101 is configured to selectively function as a peripheral device or as a central device. FIGS. 5-7 illustrate how the IMD 101 and the external device 102 connect over time.

Referring to FIG. 5, the IMD 101 and the external device 102 are illustrated as connecting two times over the course of a day, each time for set period of time (e.g., about 2 hours) before disconnecting. During most of the day, the first transceiver 416 of the IMD 101 is in the non-connectable mode (e.g., the “OFF” state) and does not transmit advertising signals or searches for advertising signals.

The second transceiver 426 is illustrated as being in a searching mode 552 all the time. Because the external device 102 may be easily recharged (because it is outside the body of the patient), saving energy may not be as significant of a concern for the external device 102 as it is for the IMD 101. Accordingly, the second transceiver 426 may be configured to be the connectable mode (e.g., in the searching mode 552) all the time. It can also be advantageous to have the second transceiver 426 be in the connectable mode all the time so that the IMD 101 can control when it connects to the external device whenever it switches into the connectable mode. The IMD 101 and the external device 102 can connect when both of the first and second transceivers 416 and 426 are in complementary connectable modes (e.g., when one is in the advertising mode and the other is in the searching mode). If the second transceiver 426 is always in a connectable mode, then a connection can be reliably initiated by switching the first transceiver 416 into the complementary connectable mode. However, the present disclosure is not limited thereto. In some other examples, the second transceiver 426 may be configured to be in the connectable mode only at select times and may otherwise be in the non-connectable mode or in a lower-power connectable mode.

At about 6 hours (6 AM) and 21 hours (9 PM), the first transceiver 416 is switched from the non-connectable mode into an advertising mode 551. After the first transceiver 416 begins advertising, the external device 102 receives the advertising signals, the second transceiver 426 may transmit a reply signal to the first transceiver 416, and the first and second transceivers 416 and 426 connect 553 (e.g., establish a communication channel) shortly after 6 hours and 21 hours, respectively. The first transceiver 416 may be switched into the advertising mode 551 in response to a set communication threshold condition occurring, examples of which are described in more detail below. The first transceiver 416 may be configured to terminate the connection 553, for example, when the first transceiver 416 is switched from the advertising mode 551 to the non-connectable mode.

Referring to FIG. 6, the activity of the first and second transceivers 416 and 426 are similar to the activity shown in FIG. 5, except that the first transceiver 416 functions as a central device in the example of FIG. 6 instead of as a peripheral device, as in the example of FIG. 5. In FIG. 6, the second transceiver 426 of the external device 102 is always in the advertising mode 551, while the first transceiver 416 of the IMD 101 is selectively switched into the searching mode 552 at certain times. The first transceiver 416 is switched from the non-connectable mode to the searching mode 552 for about half an hour at 6 hours (6 AM) and at 21 hours (9 PM). After being switched into the searching mode 552, the first transceiver 416 begins to receive advertising signals transmitted from the second transceiver 426, the first transceiver 416 transmits a reply signal to the second transceiver 426, and the first and second transceivers 416 and 426 connect 553 shortly after 6 hours and 21 hours, respectively.

FIG. 7 illustrates activity of the first and second transceivers 416 and 426 in an example where the first transceiver 416 is configured to selectively operate in an advertising mode 551 or in the searching mode 552. The IMD 101 can therefore selectively function as a peripheral device or as a central device. Similarly, the second transceiver 426 can selectively operate in the advertising mode 551 or in the searching mode 552, for example, based on the operation of the first transceiver 416. The IMD 101 and the external device 102 may be configured to coordinate the respective operations of the first and second transceivers 416 and 426 so that they operate in a complementary fashion (e.g., one operates in the advertising mode if the other is operating in the searching mode, and vice versa). The IMD 101 and the external device 102 can therefore reverse the roles of which device is operating as the peripheral device and which device is operating as the central device. This change may be initiated by one of the IMD 101 or the external device 102 transmitting a command signal to the other one of the IMD 101 or the external device 102 to reverse the roles. It can be advantageous for the IMD 101 to be controllably switched between operating in the peripheral mode or in the central mode so that the IMD 101 can connect with more types of external devices. For example, different external devices may be only operable in the peripheral mode or only in the central mode, and the IMD 101 can controllably connect with both types of external devices because it can selectively operate in either mode. Also, an IMD may be more susceptible to being hacked or accessed by hostile devices when it operates in the central mode compared to when it operates in the peripheral mode. Thus, configuring the IMD 101 to be controllably switched between operating in the peripheral mode or in the central mode can allow the IMD 101 to selectively operate in the peripheral mode, based on circumstances, to prioritize the IMD's security.

The first and second transceivers 416 and 426 connect three times for a set period shortly after 6 hours (6 AM), 13 hours (1 PM), and 21 hours (9 PM). From 0 hours to the end of the first connection 553, the second transceiver 426 operates in the searching mode 552, the first transceiver 416 begins operating in the advertising mode 551 at about 6 hours, and the first and second transceivers 416 and 426 may establish a first connection 553 shortly after 6 hours before disconnecting. The IMD 101 and the external device 102 may switch roles after the first connection 553 terminates. From the time that the first connection 553 terminates to when the second connection 553 terminates, the second transceiver 426 may operate in the advertising mode 551, the first transceiver 416 may begin operating in the searching mode 552 at about 13 hours, and the first and second transceivers 416 and 426 may establish a second connection 553 shortly after 13 hours before disconnecting. The IMD 101 and the external device 102 may switch roles again after the second connection 553 terminates. From the time that the second connection 553 terminates, the second transceiver 426 may operate in the searching mode 552, the first transceiver 416 may begin operating in the advertising mode 551 at about 21 hours, and the first and second transceivers 416 and 426 may establish a third connection 553 shortly after 21 hours.

FIGS. 5-7 describe the first transceiver 416 as being either in a connectable mode or in the non-connectable mode, but the present disclosure is not limited thereto. In some examples, the first transceiver 416 may be selectively operable in a high-power connectable mode or in a low-power mode. The low-power mode may include the non-connectable and/or a low-power connectable mode, where the low-power connectable mode uses energy at a smaller rate (e.g., uses a lower power) than the high-power connectable mode. The first transceiver 416 may be switched from the low-power mode into the high-power connectable mode in response to a communication threshold condition occurring, examples of which are described in more detail below.

In some examples, the high-power connectable mode includes a high-power advertising mode, whereby the first transceiver 416 transmits first advertising signals at a first rate, and the low-power connectable mode includes a low-power advertising mode, whereby the first transceiver 416 transmits the first advertising signals at a second rate less than the first rate. The first transceiver 416 can conserve power by transmitting the advertising signals at the second, lower rate for part of the time when communication between the IMD 101 and the external device 102 is not expected to be needed or desired, and can switch to transmitting the advertising signals at the first, higher rate when communication is needed or desired. It can be advantageous to operate the first transceiver 416 in the low-power advertising more instead of in the non-connectable mode so that the first transceiver 416 can periodically connect with the second transceiver 426, and so that the IMD 101 and the external device 102 can still perform certain daily tasks (e.g., exchange information and/or instructions) in the background while still conserving energy. Operating the first transceiver 416 in the low-power advertising mode (or in the non-connectable mode) can also improve security of the IMD 101 by reducing the amount of time in which the IMD 101 is discoverable to external devices, which can reduce the likelihood of attempts by unauthorized or malicious devices to connect to the IMD 101.

In some examples, the high-power connectable mode may include a high-power searching mode, and the low-power connectable mode may include a low-power searching mode. The first transceiver 416, when operating in the low-power searching mode, may perform a coarse filter on incoming signals to identify an advertising signal, or may simply search for any incoming signal. The first transceiver 416, when operating in the high-power searching mode, may perform a finer filter to identify an advertising signal with a higher confidence level than when using the coarse filter.

In some examples, the IMD 101 is configured to perform one or more of its core functions (e.g., providing stimulation, sensing one or more physiological parameters or conditions, and/or discharging a medical substance) while the first transceiver 416 operates in the low-power mode. The IMD 101 may be configured to perform its core function(s) while the first transceiver 416 operates in the high-power connectable mode. The IMD 101 can therefore conserve power by switching the first transceiver 416 from the high-power connectable mode to the low-power mode, even while it continues to perform one or more of its core functions. For example, the IMD 101 may be configured to provide stimulation to the vagus nerve over the course of the day to treat epilepsy, and may switch the first transceiver 416 into the high-power connectable mode only occasionally when communication between the IMD 101 and the external device 102 is needed or desired.

The first MCU 411 may be configured to selectively cause the first transceiver 416 to operate in the high-power connectable mode or in the low-power mode based on first data sensed by the one or more sensors 461. For example, the first MCU 411 may be configured to cause the first transceiver 416 to switch from operating in the low power mode to operating in the high-power connectable mode in response to determining (e.g., based on the first data) that a communication threshold condition has been determined (e.g., determined to be presented or to have occurred). The first MCU 411 may be configured to analyze the first data to determine whether the communication threshold condition has been determined.

In some examples, the communication threshold condition may include the initiation of charging of a rechargeable battery of the IMD 101 (e.g., via an inductive charging field generated by a wireless charger outside of the patient's body), which may be determined by the first MCU 411. The communication threshold condition may include a state of charge of the rechargeable battery of the IMD 101 falling below a threshold value, which may be determined by the first MCU 411.

The one or more sensors 461 may be on the IPG 110 and/or on a lead coupled to the IPG 110. In some other examples, the one or more sensors 461 may be on a sensor device that is separate from the IMD 101. The separate sensor device may be worn on the patient or implanted in the patient and may be configured to wirelessly transmit the first data to the IMD 101.

In some examples, the one or more sensors 461 are configured to sense physiological data. The first MCU 411 may be configured to analyze the sensed physiological data to determine when a physiological condition has been determined, and the first MCU 411 may be configured to cause the first transceiver 416 to being operating in the high-power connectable mode (e.g., to switch from the low-power mode to the high-power connectable mode) in response to determining that the physiological condition has been determined.

For example, the one or more sensors 461 may include at least one of an electroencephalography (EEG) sensor, an electrocardiography (ECG) sensor, a respiration sensor, and/or a pressure sensor. The respiration sensor may be configured to measure one or more respiration parameters, such as respiration frequency, respiration intensity, and/or respiration rate variability. The pressure sensor may be configured, for example, to measure a fill level of the patient's bladder. The physiological condition may include a seizure, an apneic event, the patient being asleep, and/or a fill level of the patient's bladder being above a threshold fill level.

In some examples, the IMD 101 may be configured to treat seizures in an epileptic patient, for example, by providing stimulation to the vagus nerve, and the one or more sensors 461 may include the EEG sensor and/or the ECG sensor for detecting whether a seizure is occurring or is about to occur. The IMD 101 may be configured to provide the stimulation generally autonomously over the course of the day without patient involvement, and the IMD 101 may only occasionally need to communicate with the external device 102. The IMD 101 may normally operate the first transceiver 416 in the low-power mode and may switch the first transceiver 416 into the high-power connectable mode when the IMD 101 needs to communicate with the external device 102. For example, the first MCU 411 may be configured to switch the first transceiver 416 into the high-power connectable mode in response to determining, based on physiological data sensed by the EEG sensor, the ECG sensor, and/or the respiration sensor that the patient is experiencing, or is about to experience, a seizure. It can be desirable for the IMD 101 to communicate with the external device 102 to, for example, inform the external device 102 that the patient is experiencing a seizure. The external device 102 can then alert, for example, nearby individuals (e.g., family members or caregivers of the patient) or authorities (e.g., the police or a hospital) that the patient is experiencing seizures. The IMD 101 can also transmit to the external device 102 physiological data sensed by the IMD 101.

In some examples, the IMD 101 may be configured to treat patients with sleep apnea by, for example, by providing stimulation (e.g., to the hypoglossal nerve) to open the patient's airway when the patient is experiencing an apneic event. An apneic event may occur when the patient's airway at least partly closes and the patient struggles to breathe, and providing stimulating, for example, to the hypoglossal nerve, can cause the patient's airway to open up again. The one or more sensors 461 may include the respiration sensor to measure the one or more respiration parameters. The first MCU 411 may be configured to switch the first transceiver 416 into the high-power connectable mode in response to determining, based on the one or more respiration parameters, that the patient is experiencing an apneic event.

In some examples, the IMD 101 is configured to treat patients with under-active bladder syndrome, in which the patient has trouble fully emptying his or her bladder when urinating. The IMD 101 may be configured to provide stimulation to the bladder to effectuate a more complete evacuation of the bladder. The one or more sensors 461 may include the pressure sensor configured to detect a pressure level and/or a fill level of the bladder. This pressure measurement can be used to determine that the fill level or pressure level of the bladder is above an upper threshold level (indicating that the patient should urinate soon) and/or that the fill level or pressure level is below a lower threshold level (indicating that urination can be stopped). The first MCU 411 may be configured to switch the first transceiver 416 from the low-power mode to the high-power connectable mode in response to determining, based on the physiological data sensed by the pressure sensor, that the fill level or pressure level is above the upper threshold level. The communication channel 195 between the IMD 101 and the external device 102 can then be established, and the patient can initiate stimulation to empty his or her bladder by sending a command signal to the IMD 101 via the external device 102. It can also be desirable for the IMD 101 to transmit physiological data (e.g., fill or pressure levels of the bladder before, during, and after evacuation) sensed by the IMD 101 to the external device 102 so that the patient (or the patient's physician) can evaluate the physiological data.

The first MCU 411 may be configured to keep the first transceiver 416 in the high-power connectable mode for a set time after switching the first transceiver 416 into the high-power connectable mode and/or the first MCU 411 may be configured to switch the first transceiver 416 back into the low-power mode from the high-power connectable mode in response to additional data sensed by the one or more sensors 461. As discussed above, it can be desirable to reduce the time in which the first transceiver 416 operates in the high-power connectable mode in order to conserve energy in the IMD 101. Accordingly, in some examples, the first MCU 411 may switch the first transceiver 416 into the high-power connectable mode for only the set time period before switching the first transceiver 416 back into the low-power mode. This set time period may be sufficiently long to allow the IMD 101 to transmit information (e.g., to transmit sensed physiological data, battery state of charge, etc.) to the external device 102 and/or for the patient to transmit instructions or information to the IMD 101 via the external device 102. In some examples, the first MCU 411 may keep the first transceiver 416 in the high-power connectable mode until certain physiological conditions (e.g., the end of a seizure, apneic event, etc.) are determined to be present or to have occurred and, in some examples, until a set time thereafter.

The first MCU 411 may be configured to identify a wake-up signal in the first data sensed by the one or more sensors 461. The wake-up signal may be a signal indicating the patient's desire or need to communicate between the IMD 101 and the external device 102. The first MCU 411 may be configured to switch the first transceiver 416 from the low-power mode to the high-power connectable mode in response to determining, based on the first data, that the IMD 101 has receive the wake-up signal.

In some examples, the first MCU 411 may be configured to maintain the first transceiver 416 in the high-power connectable mode for a set period of time after switching the first transceiver 416 into the high-power connectable mode. In some examples, the first MCU 411 may be configured to keep the first transceiver 416 in the high-power connectable mode until it identifies a turn-off signal in subsequent data sensed by the one or more sensors 461, or until a set time period has lapsed from when the first MCU 411 identified the turn-off signal.

In some examples, the one or more sensors 461 may include an inertial measurement unit (IMU) and/or an accelerometer. The IMU and/or the accelerometer may be configured to detect taps against the body of the patient that the IMD 101 is implanted in. The first MCU 411 may be configured to determine, based on data sensed by the IMU and/or the accelerometer, that a first sequence of taps has occurred against the patient's body. The first sequence of taps (e.g., a double tap, a triple tap, or any other sequence of taps) may be recognized by the first MCU 411 as the wake-up signal. The first sequence of taps, or a second sequence of taps different from the first sequence of taps, may be recognized by the first MCU 411 as the turn-off signal.

In some examples, the first MCU 411 may be configured to cause the IMD 101 to perform one or more core functions in response to identifying an activation signal from among the first data sensed by the one or more sensors 461. For example, in response to identifying the activation signal, the first MCU 411 may be configured to provide a stimulation boost (e.g., to the vagus nerve to treat a current or impending seizure), to provide stimulation to the bladder to empty the patient's bladder, or to discharge a medical substance (e.g., insulin via an insulin pump).

The activation signal may be the same as, or different from, the wake-up signal. In some examples, the activation signal is different from the wake-up signal. In some such examples, the patient can, via the wake-up signal, selectively connect the IMD 101 and the external device 102 without activating the one or more core functions, and the patient can, via the activation signal, selectively activate the one or more core functions without connecting the IMD 101 and the external device 102. In some other examples, the wake-up signal is the same as the activation signal, and the first MCU 411 may be configured, in response to identifying the activation signal, to both activate the one or more core functions of the IMD 101 and to switch the first transceiver 416 into the high-power connectable mode. The first transceiver 416 may then be left in the high-power connectable mode (e.g., for a set time period) before being switched back into the low-power mode. For example, a patient with under-active bladder syndrome can use a sequence of taps to both initiate urination and also connect the IMD 101 and the external device 102 for a set time period.

In some examples, the first MCU 411 is configured to determine, based on the first data sensed by the one or more sensors 461, information about the patient's bodily orientation, for example, whether the patient's torso is in an upright position (indicating standing or sitting) or a horizontal position (e.g., a lying down position). The first MCU 411 may be configured to selectively cause the first transceiver 416 to operate in the high-power connectable mode or in the low-power mode based on the patient's bodily orientation, for example, based on whether the patient is in the upright position, is in the horizontal position, has moved from the horizontal position to the upright position, or has moved from the upright position to the horizontal position. This information may indicate that the patient is getting ready to go to sleep (e.g., when the patient is in the horizontal position) or is awake (e.g., when the patient is in the upright position), and it may be desirable at these times to initiate connection (e.g., temporarily for a set time period) between the IMD 101 and the external device 102 and/or to switch the first transceiver 416 into the low-power mode to conserve energy while the patient sleeps.

The one or more sensors 461 may include at least one of an inertial measurement unit (IMU) or an accelerometer that are configured to sense information about the patient's bodily orientation.

In some examples, the first MCU 411 is configured to cause the first transceiver 416 to begin operating in the high-power connectable mode in response to determining, based on the first data collected by the one or more sensors 461, that the patient has moved between the horizontal position and the upright position.

For example, the first MCU 411 may initiate a communication shut-down operation in response to determining that the patient has moved from the upright position to the horizontal position and, in some examples, after the patient remains in the horizontal position for a set period of time. The communication shut-down operation may include switching the first transceiver 416 from operating in the high-power connectable mode into the low-power mode within a set time period after the first MCU 411 determines that the patient has moved from the upright position to the horizontal position. When the patient moves from the upright position to the horizontal position, this may indicate that the patient is getting ready to go to sleep. While the patient is asleep, communication between the IMD 101 and the external device 102 may not be needed or desirable, and so it can be advantageous to operate the first transceiver 416 in the low-power mode to conserve energy while the patient sleeps. Waiting for the patient to remain in the horizontal position for a set time period before initiating the communication shut-down operation may help to avoid false flags where the patient briefly lies down for reasons other than going to sleep.

In some examples, the first MCU 411 may initiate a communication start-up operation in response to determining that the patient has moved from the horizontal position to the upright position and, in some examples, after the patient remains in the upright position for a set period of time. The communication start-up operation may include switching the first transceiver 416 from the low-power mode into the high-power connectable mode within a first set time period after the first MCU 411 determines, based on the data sensed by the one or more sensors 461, that the patient has moved from the horizontal position to the upright position. This movement may indicate that the patient has woken up. Because the patient may be more likely to communicate between the IMD 101 and the external device 102 while awake, it may be advantageous to switch the first transceiver 416 into the high-power connectable mode at this time. Establishing the communication channel between the IMD 101 and the external device 102 when the patient wakes up can also allow the IMD 101 to transmit information (e.g., sensed physiological data while the patient was asleep, battery state of charge, etc.) to the external device 102 at this time.

In some cases, the patient may not need or want to communicate between the IMD 101 and the external device 102 very often even while awake. For example, the IMD 101 may be configured to provide stimulation to the vagus nerve throughout the day to treat epilepsy in a generally autonomous manner without the patient's involvement. Accordingly, in some examples, the communication start-up operation may include causing the first transceiver 416 to continue to operate in the high-power connectable mode for a set second time period after the first transceiver 416 is switched from the low-power mode to the high-power connectable mode, and switching the first transceiver 416 from the high-power connectable mode to the low-power mode after the second time period ends. This can allow for a temporary connection between the IMD 101 and the external device 102 when the patient wakes up to allow the IMD 101 to transmit information to the external device 102 and/or for the patient to transmit instructions to the IMD 101 via the external device 102. After the temporary connection, the first transceiver 416 can be transitioned back into the low-power mode to conserve energy during the day.

In some examples, the one or more sensors 461 may include the respiration sensor with or without the IMU and/or accelerometer. The respiration sensor may be configured to measure the one or more respiration parameters (e.g., respiration frequency, respiration intensity, and/or respiration rate variability) in the patient, which may indicate whether the patient is awake or asleep. The first MCU 411 may be configured to selectively operate the first transceiver 416 in the low-power mode or in the high-power connectable mode based on the detected respiration levels with or without the information about the patient's bodily orientation.

For example, the first MCU 411 may be configured to initiate the communication shut-down operation in response to determining that a respiration parameter is below a threshold value. In some examples, the first MCU 411 may be configured to initiate the communication shut-down operation in response to determining both that the respiration is below the threshold value and that the patient is in the horizontal position. In some examples, the first MCU 411 may be configured to initiate the communication start-up operation in response to determining that the respiration parameter has risen above a second threshold value, or in response to determining both that the respiration parameter is above the second threshold value and that the patient is in the upright position. Selectively operating the first transceiver 416 based on the one or more respiration parameters can provide an alternative method for determining when the patient is going to sleep or waking up, or it can be used in combination with the patient's bodily orientation information to improve accuracy of determining when the patient is asleep or awake.

In some examples, the first MCU 411 may be configured to collect second data, based on the time determined by the clock 417, about the time(s) of day that the first transceiver 416 is operated in the high-power connectable mode. The first MCU 411 may be configured to then selectively operate the first transceiver 416 in the high-power connectable mode or in the low-power mode based on the current time of day and the second data. In some cases, the time(s) of the day at which the IMD 101 and the external device 102 connect may generally be cyclical. For example, the patient may typically want the IMD 101 and the external device 102 to be connected before the patient goes to bed and/or when the patient gets up in the morning. Or the patient may reliably transmit the wake-up signal to the IMD 101 at set time(s) of the day, or the patient may experience a physiological condition at set time(s) of day that prompt the first MCU 411 to switch the first transceiver 416 into the high-power connectable mode. By selectively operating the first transceiver 416 in the higher power connectable mode or in the low-power mode based on the second data, the IMD 101 and the external device 102 can be reliably connected at the time(s) of day when it is needed or desirable for the IMD 101 and the external device 102 to be connected. The second data may be periodically updated with subsequent data about the time(s) of day when the IMD 101 and the external device 102 connect.

In some examples, the second data may include data about the time(s) of day when the set physiological condition is determined by the first MCU 411 to occur, the time(s) of day that the first MCU 411 detects the wake-up signal, and/or the time(s) of day when the first MCU 411 determines that the patient is in the horizontal position. This data may be compiled over the course of several days (e.g., at least 2, 5, 7, 10, or 30 days). The first MCU 411 may be configured to selectively operate the first transceiver 416 in the high-power connectable mode or in the low-power mode based on the second data with or without the subsequently sensed first data.

For example, the first MCU 411 may be configured to selectively operate the first transceiver 416 in the high-power connectable mode or in the low-power mode based on the patient's current bodily orientation and the second data about time(s) of day when the patient is usually asleep. Using this combination of data, the first MCU 411 can more accurately determine whether the patient is asleep or awake.

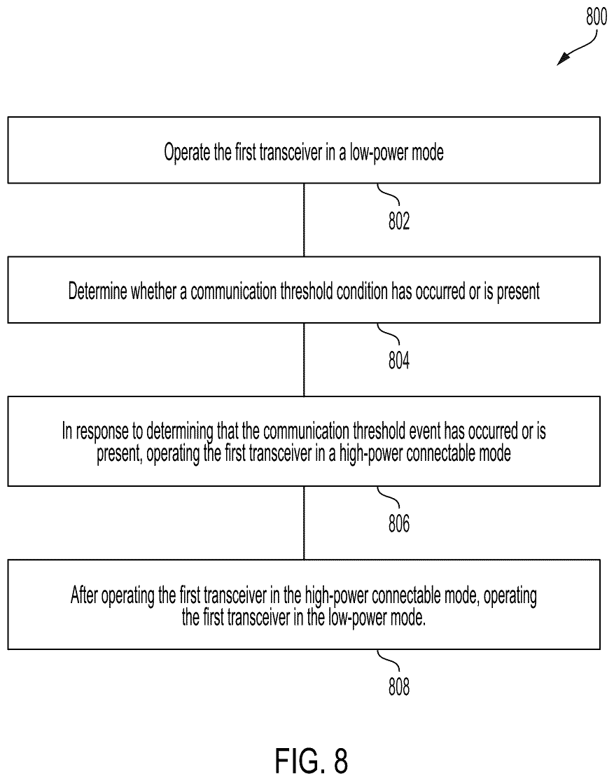

FIG. 8 illustrates a method 800 for operating an IMD, according to some examples. The IMD may include a first transceiver, and the IMD and the first transceiver may have features similar to, or the same as, features of the IMD 101 and of the first transceiver 416, respectively.

The method 800 may include a first process 802 of operating the operating the first transceiver in a low-power mode. The low-power mode may have features similar to, or the same as, features of the low power mode described herein with reference to the medical system of FIGS. 1-4. For example, the low-power mode may include the non-connectable mode, the low-power advertising mode, or the low-power searching mode.

The method 800 may include a second process 804, after the first process 802, of determining whether a communication threshold condition has occurred or is present. The communication threshold condition may be, for example, any communication threshold condition described herein. For example, the communication threshold condition may include a physiological condition, receiving a wake-up signal by the IMD, a change in position of the body of a patient that the IMD is implanted in, or a time of day (e.g., as measured by a clock of the IMD) being within a set range (e.g., within 9 PM to 10 PM), etc.

The method 800 may include a third process 806 of operating the first transceiver in a high-power connectable mode in response to determining that the communication threshold event has occurred or is present. The high-power connectable mode may include features similar to, or the same as, features of the high-power connectable mode described herein with reference to the medical system of FIGS. 1-4. For example, the high-power connectable mode may include the high-power advertising more or the high-power searching mode.

The method 800 may include a fourth process 808 of operating the first transceiver in the low-power mode again after the first transceiver has been operating in the high-power connectable mode. For example, the first transceiver may be switched from operating in the high-power connectable mode to operating in the low-power mode after the first transceiver has been operating in the high-power connectable mode for a set time period.

Although some methods for operating a medical system have been discussed with reference to FIG. 8, the present disclosure is not limited thereto. Medical systems, and processes performed by such systems, have been described herein with reference to the drawings, and the present disclosure includes all methods for operating a medical system that include any combination of such processes in any suitable order.

Features from an embodiment, or from multiple embodiments, described in the present disclosure may be combined with each other, partially or entirely, and may be technically interlocked and operated in various ways, and the embodiments described herein may be implemented independently of each other or in conjunction with each other.

Although specific embodiments are described herein, the scope of the technology is not limited to those specific embodiments. Moreover, while different embodiments may be described separately, such embodiments and examples may be combined with one another in implementing the technology described herein. One skilled in the art will recognize other embodiments or improvements that are within the scope and spirit of the present technology. Therefore, the specific elements, features, and processes are disclosed only as example embodiments. The scope of the technology is defined by the following claims and any equivalents thereof.

What is claimed is:

1. An implantable medical device (IMD), comprising:
a first transceiver configured to operate in a first advertising mode, whereby the first transceiver transmits a first advertising signal at a first rate;
one or more sensors configured to sense first data;
a memory storing instructions; and
a microcontroller circuit operatively coupled to the first transceiver, the microcontroller circuit being configured, in response to executing the instructions stored in the memory, to selectively cause the first transceiver to begin operating in the first advertising mode based on the first data.

2. The IMD of claim 1, wherein the microcontroller circuit is configured to selectively cause the first transceiver to operate:
in the first advertising mode; or
in a second advertising mode, whereby the first transceiver does not transmit an advertising signal or transmits the first advertising signal at a second rate less than the first rate.

3. The IMD of claim 2, comprising:
an implantable pulse generator (IPG) comprising the first transceiver, the microcontroller circuit, the memory, and a driver configured to generate an electrical current;
a lead electrically coupled to the IPG; and
a stimulation electrode on the lead and electrically coupled to the driver through the lead,
wherein the microcontroller circuit is operatively coupled to the driver and is configured to cause the driver to generate the electrical current while the first transceiver operates in the second advertising mode.

4. A medical system, comprising:
the IMD of claim 1; and
an external device comprising a second transceiver configured to operate in a first searching mode, whereby the second transceiver searches for an advertising signal,
wherein the IMD and the external device are configured to establish a communication channel between the IMD and the external device in response to the second transceiver receiving the first advertising signal and transmitting a reply signal to the first transceiver.

5. The medical system of claim 4, wherein the medical system is configured to switch between operating in:
an IMD peripheral mode, whereby the first transceiver operates in the first advertising mode and the second transceiver operates in the first searching mode; or
an IMD central mode, whereby the first transceiver operates in a second searching mode and the second transceiver operates in a second advertising mode,
wherein the first transceiver is configured to search for an advertising signal when operating in the second searching mode, and
wherein the second transceiver is configured to transmit second advertising signals when operating in the second advertising mode.

6. The medical system of claim 5, wherein the first data comprises physiological data, and
wherein the microcontroller circuit is configured, when the first transceiver operates in the second searching mode, to cause the first transceiver to search for the advertising signal in response to the microcontroller circuit determining, based on the physiological data, that a physiological condition has occurred or is present.

7. The medical system of claim 4, wherein the first transceiver comprises a first Bluetooth radio, and the external device comprises a second Bluetooth radio.

8. The IMD of claim 1, wherein the first data comprises physiological data, and the microcontroller circuit is configured to selectively cause the first transceiver to begin operating in the first advertising mode in response to determining, based on the physiological data, that a physiological condition has occurred or is present.

9. The IMD of claim 8, wherein the one or more sensors comprise an electroencephalography (EEG) sensor.

10. The IMD of claim 8, wherein the one or more sensors comprise an electrocardiogramansor.

11. The IMD of claim 8, wherein the physiological condition comprises a seizure.

12. The IMD of claim 8, wherein the physiological condition comprises an apneic event.

13. The IMD of claim 1, wherein the microcontroller circuit is configured to selectively cause the first transceiver to begin operating in the first advertising mode in response to identifying a wake-up signal in the first data.

14. The IMD of claim 13, wherein the one or more sensors comprise at least one of an inertial measurement unit (IMU) or an accelerometer.

15. The IMD of claim 13, wherein identifying the wake-up signal comprises determining, based on the first data, that a first sequence of taps has occurred against a body of a patient that the IMD is implanted in.

16. The IMD of claim 1, wherein the microcontroller circuit is configured to selectively cause the first transceiver to begin operating in the first advertising mode in response to determining, based on the first data, that a patient that the IMD is implanted in is in an upright position or a horizontal position.

17. The IMD of claim 16, wherein the one or more sensors comprise at least one of an inertial measurement unit (IMU) or an accelerometer.

18. The IMD of claim 16, wherein the microcontroller circuit is configured to selectively cause the first transceiver to begin operating in the first advertising mode in response to determining that the patient has moved between the horizontal position and the upright position.

19. The IMD of claim 18, wherein the microcontroller circuit is configured to cause the first transceiver to begin a communication shut-down operation in response to determining that the patient has moved from the upright position to the horizontal position, the communication shut-down operation comprising the first transceiver stopping its operating in the first advertisement mode within a set first time period after the microcontroller circuit determines that the patient has moved from the upright position to the horizontal position.

20. The IMD of claim 18, wherein the microcontroller circuit is configured to cause the first transceiver to begin a communication activation operation in response to determining that the patient has moved from the horizontal position to the upright position, the communication activation operation comprising the first transceiver beginning its operating in the first advertising mode within a first set time period after the microcontroller circuit determines that the patient has transitioned from the horizontal position to the upright position.

21. The IMD of claim 20, wherein the communication activation operation comprises causing the first transceiver to operate in the first advertising mode for a second time period, and to stop operating in the first advertising mode after the second time period.

22. The IMD of claim 16, wherein the first data comprises a respiration level.

23. The IMD of claim 22, wherein the microcontroller circuit is configured to selectively cause the first transceiver to begin operating in the first advertising mode based on whether the respiration level is below a threshold respiration level and whether the patient is in the horizontal position.

24. The IMD of claim 16, comprising a clock configured to determine a time of day, wherein the microcontroller circuit is configured to collect second data about time(s) of day when the patient is in the horizontal position, and to selectively cause the first transceiver to begin operating in the first advertising mode based on the time of day and the second data.

25. The IMD of claim 1, comprising a clock configured to determine a time of day,
wherein the microcontroller circuit is configured to selectively cause the first transceiver to operate in the first advertising mode or in a second advertising mode, whereby the first transceiver does not transmit an advertising signal or transmits the first advertising signal at a second rate less than the first rate, and
wherein the microcontroller circuit is configured to collect second data about time(s) of day when the first transceiver is operated in the first advertising mode, and to selectively cause the first transceiver to begin operating in the first advertising mode based on the time of day and the second data.

26. An implantable medical device (IMD), comprising:
a first transceiver configured to operate in a high-power connectable mode and in a low-power mode;
a memory storing instructions; and
a microcontroller circuit operatively coupled to the first transceiver, the microcontroller circuit being configured, in response to executing the instructions stored in the memory, to selectively cause the first transceiver to operate in the high-power connectable mode or in the low-power mode.

27. The IMD of claim 26, wherein the first transceiver is configured, when operating in the high-power connectable mode, to transmit a first advertising signal at a first rate, and
wherein the first transceiver is configured, when operating in the low-power mode, to not transmit an advertising signal or to transmit the first advertising signal at a second rate less than the first rate.

28. The IMD of claim 26, wherein the first transceiver is configured, when operating in the high-power connectable mode, to search for advertising signals and to use energy at a first rate, and
wherein the first transceiver is configured, when operating in the low-power mode, to not search for advertising signals or to search for advertising signals while using energy at a second rate less than the first rate.

29. The IMD of claim 26, wherein the microcontroller circuit is configured to cause the IMD to perform one or more core operations while the first transceiver operates in the low-power mode, the one or more core operations comprising at least one of providing stimulation, sensing physiological data, or discharging a medical substance.

30. The IMD of claim 26, wherein IMD comprises:
an implantable pulse generator (IPG) comprising the first transceiver, the microcontroller circuit, the memory, and a driver configured to generate an electrical current;
a lead electrically coupled to the IPG; and
a stimulation electrode on the lead and electrically coupled to the driver.

31. The IMD of claim 26, comprising one or more sensors,
wherein the microcontroller circuit is configured to cause the first transceiver to transition from operating in the low-power mode to operating in the high-power connectable mode in response to determining, based on physiological data sensed by the one or more sensors, that a physiological condition has occurred or is present.

32. The IMD device of claim 31, wherein the one or more sensors comprise at least one of an electroencephalography (EEG) sensor or an electrocardiography (ECG) sensor.

33. The IMD device of claim 31, wherein the physiological condition comprises at least one of a seizure or an apneic event.

34. The IMD device of claim 26, comprising one or more sensors,
wherein the microcontroller circuit is configured to cause the first transceiver to transition from operating in the low-power mode to operating in the high-power connectable mode in response to identifying a wake-up signal in data sensed by the one or more sensors.

35. The IMD of claim 34, wherein the one or more sensors comprise at least one of an inertial measurement unit or an accelerometer.

36. The IMD of claim 34, wherein the identifying the wake-up signal comprises determining that a first sequence of taps has occurred against a body of a patient that the IMD is implanted in.

37. The IMD of claim 36, comprising:
an implantable pulse generator (IPG) comprising the first transceiver, the microcontroller circuit, the memory, and a driver configured to controllably provide an electrical current;
a lead electrically coupled to the IPG; and
a stimulation electrode on the lead and electrically coupled to the driver,
wherein the microcontroller circuit is operatively coupled to the driver and configured to cause the driver to provide the electrical current to the stimulation electrode in response to determining that the first sequence of taps has occurred against the body.

38. The IMD of claim 36, wherein the IMD comprises:
an implantable pulse generator (IPG) comprising the first transceiver, the microcontroller circuit, the memory, and a driver configured to controllably provide an electrical current;
a lead electrically coupled to the IPG; and
a stimulation electrode on the lead and electrically coupled to the driver,
wherein the microcontroller circuit is operatively coupled to the driver and configured to cause the driver to provide the electrical current in response to determining that a second sequence of taps against the body, different from the first sequence of taps, has occurred.

39. The IMD of claim 26, comprising one or more sensors,
wherein the microcontroller circuit is configured to cause the first transceiver to transition between operating in the low-power mode and operating in the high-power connectable mode in response to determining, based on data sensed by the one or more sensors, that a patient that the IMD is implanted in has moved between a horizontal position and an upright position.

40. The IMD of claim 39, wherein the one or more sensors comprise at least one of an inertial measurement unit or an accelerometer.

41. The IMD of claim 39, wherein the microcontroller circuit is configured, in response to determining that the patient has moved from the upright position to the horizontal position, to cause the first transceiver to operate in the high-power connectable mode for a first time period and to transition from operating in the high-power connectable mode to operating in the low-power mode after the first time period.

42. The IMD of claim 39, wherein the microcontroller circuit is configured, in response to determining that the patient has moved from the horizontal position to the upright position, to cause the first transceiver to transition from operating in the low-power mode to operating in the high-power connectable mode.

43. The IMD of claim 26, comprising a clock configured to determine a time of day, wherein the microcontroller circuit is configured to selectively cause the first transceiver to operate in the high-power connectable mode based on the time of day.

44. The IMD of claim 43, wherein the microcontroller circuit is configured to collect second data about what time(s) of day the first transceiver is operated in the high-power connectable mode, and to selectively cause the first transceiver to operate in the high-power connectable mode based on the second data.

45. The IMD of claim 44, comprising one or more sensors,
wherein the microcontroller circuit is configured to selectively cause the first transceiver to operate in the high-power connectable mode in response to at least one of:
determining a physiological condition based on physiological data sensed by the one or more sensors;
identifying a wake-up signal in data sensed by the one or more sensors; or
determining that a set change has occurred in a position of a patient's body that the IMD is implanted in.

46. A medical system, comprising:
the IMD of claim 26; and
an external device comprising a second transceiver configured to establish a communication channel with the first transceiver when the first transceiver operates in the high-power connectable mode.

47. The medical system of claim 46, wherein the first and second transceivers are not configured to establish the communication channel when the first transceiver operates in the low-power mode.

48. A method for operating an implantable medical device (IMD) comprising a first transceiver, the method comprising:
operating the first transceiver in a low-power mode;
determining that a communication threshold condition has occurred or is present; and
in response to determining that the communication threshold condition has occurred or is present, operating the first transceiver in a high-power connectable mode, whereby the first transceiver transmits advertising signals at a first rate or searches for advertising signals, the first transceiver uses energy at a higher rate when operating in the high-power connectable mode than when operating in the low-power mode.

49. The method of claim 48, wherein, when the first transceiver operates in the low-power mode, the first transceiver does not transmit advertising signals and does not search for advertising signals.

50. The method of claim 48, wherein the determining that the communication threshold condition has occurred or is present comprises determining that a physiological condition has occurred or is present.

51. The method of claim 48, wherein the determining that the communication threshold condition has occurred or is present comprises identifying a wake-up signal in data sensed by the IMD.

52. The method of claim 48, wherein the determining that the communication threshold condition has occurred or is present comprises determining that a set change has occurred in a position of a patient's body that the IMD is implanted in.

53. The method of claim 48, wherein the determining that the communication threshold condition has occurred or is present comprises determining that the time of day is within a set range.
